(19) United States
(12) Patent Application Publication
(10) Pub. No.: US 2020/0010826 A1
(43) Pub. Date: Jan. 9, 2020

(54) SIMULTANEOUS SEPARATION AND ACTIVATION OF T CELLS FROM BLOOD PRODUCTS WITH SUBSEQUENT STIMULATION TO EXPAND T CELLS

(71) Applicant: BIOMAGNETIC SOLUTIONS LLC, State College, PA (US)

(72) Inventors: Paul A. LIBERTI, State College, PA (US); Dustin W. RITTER, State College, PA (US); Todor R. KHRISTOV, State College, PA (US)

(73) Assignee: Biomagnetic Solutions LLC, State College, PA (US)

(21) Appl. No.: 16/320,531

(22) PCT Filed: Jul. 26, 2017
(86) PCT No.: PCT/US2017/043855

§ 371 Date: Jan. 25, 2019

Related U.S. Application Data
(60) Provisional application No. 62/366,696, filed on Jul. 26, 2016.

(51) Int. Cl. C12N 13/00; C12N 5/0783

(52) U.S. Cl. CPC C12N 13/00; C12N 2501/998; C12N 2501/51; C12N 5/0636

(57) ABSTRACT

Embodiments disclosed herein relate to methods for purifying, activating, and expanding T cells, and subsets thereof.

### CROSS REFERENCE TO RELATED APPLICATIONS

This application claims priority to U.S. Provisional Patent Application No. 62/366,696, filed Jul. 26, 2016, which is hereby incorporated by reference in its entirety.

### FIELD

Embodiments disclosed herein relate to methods for purifying, activating, and expanding T cells, and subsets thereof.

### BACKGROUND

Adoptive immunotherapy holds great potential as a therapeutic modality for the treatment of a variety of diseases including cancer and chronic viral infections. Based on some extraordinary clinical successes with T cells genetically engineered to express a chimeric antigen receptor (CAR-T cells) directed to CD19 that oblate B cell malignancies, as well as the intense search for targetable unique tumor markers on solid tumors, the purification, activation, expansion and genetic modification of immune cells—particularly T cells—has become an area of significant interest. At present, an extraordinary level of effort is being put forth in each of these areas based on the early promise of actually curing various cancers. However, due to the significant cost and complexities of delivering such therapies, there is a need to develop methods that are simple, economical, efficient, and cGMP compliant. Embodiments disclosed herein satisfy these needs as well as others.

### BRIEF SUMMARY

Embodiments disclosed herein provide for the simultaneous separation and activation of T cells, or subsets thereof.

In some embodiments, methods of simultaneously separating and activating a population of T cells, or subsets thereof, are provided. In some embodiments, the method comprises a) incubating a sample comprising a population of labeled magnetic particles, with at least one antibody that binds to a T-cell cell surface protein and activates the T cell, and a blood product; b) applying a magnetic force to the sample; and c) separating the cells that are bound to the magnetic particles from the cells that are not bound to the magnetic particles, wherein the labeled magnetic particles are labeled with a common-capture reagent.

In some embodiments, methods of simultaneously separating and activating a population of T cells, or subsets thereof, are provided. In some embodiments, the methods comprising a) incubating a sample comprising: a blood product; a population of non-magnetic particles bound to at least one first antibody that binds to a T-cell surface protein and activates a T cell in the blood product; a population of magnetic particles bound to at least one second antibody that binds to a cell surface protein of a cell in the blood product that is not a T cell, wherein the second antibody does not bind to the non-magnetic particles; b) applying a magnetic force to the sample; c) separating the cells that are bound to the magnetic particles from the cells that are not bound to the magnetic particles; and d) optionally culturing the cells that are not bound to the magnetic particles to expand the population of cells.

In some embodiments, methods of simultaneously separating and activating a sub-population of T cells, or subsets thereof, are provided, the method comprising: a) incubating a sample comprising: a blood product; a population of magnetic particles bound to at least one first antibody that binds to a cell surface protein of a desired sub-population of cells in the blood product; a population of non-magnetic particles bound to at least one second antibody that binds to and activates the desired sub-population of cells in the blood product, wherein the second antibody does not bind to the magnetic particles; b) applying a magnetic force to the sample; c) separating the cells that are bound to the magnetic particles from the cells that are not bound to the magnetic particles; and d) optionally culturing the cells that are bound to the magnetic particles to expand the sub-population of cells.

In some embodiments, methods of simultaneously separating and activating a sub-population of T cells, or subsets thereof, are provided, the method comprising: a) incubating a sample comprising: a blood product; a population of magnetic particles bound to at least one first antibody that binds to the cells not in a desired sub-population of cells in the blood product; and a population of non-magnetic particles bound to at least one second antibody that binds to a cell surface protein of and activates the desired sub-population of cells in the blood product, wherein the second antibody does not bind to the magnetic particles; b) applying a magnetic force to the sample; c) separating the cells that are bound to the magnetic particles from the cells that are not bound to the magnetic particles; and d) optionally culturing the cells that are not bound to the magnetic particles to expand the sub-population of cells.

### DETAILED DESCRIPTION

As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “an antibody” is a reference to one or more antibodies and equivalents thereof known to those skilled in the art, and so forth.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “about” means plus or minus 10% of the numerical value of the number with which it is being used. Therefore, about 50% means in the range of 45% to 55%. As used herein, when referencing a range, the term “about” modifies both ends of the range even if the term is not used explicitly. For example, the phrase “about 4:1” means a ratio of “about 4 to about 1”. Additionally, where the term “about” is used, the amount or range is also provided without the term “about.” For example, about 2:1 also provides for a ratio of 2:1.

“Antibody”, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody molecule refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs).

The term “antibody” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, Md., Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66- 94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference.

As used herein, the term “optional” or “optionally” means that the subsequently described structure, event or circumstance may or may not occur, and that the description includes instances where the event occurs and instances where it does not.

The terms “at least one first antibody” and “at least one second antibody” are used throughout the present specification in various embodiments. These terms can refer to a single antibody or a population of antibodies being used for their intended purpose. For example, in some embodiments, an antibody is used as a separation antibody. That is, a separation antibody is used to separate away one population of cells from another. The separation antibody can be a single antibody or a plurality of antibodies, such as those that are described herein. The plurality of antibodies can be modified to include certain antibodies depending on the desired separation. Furthermore, the antibodies disclosed herein are non-limiting examples and other antibodies can also be used or substituted.

Without being bound to any particular theory, the embodiments provided for herein are a result of discoveries made during the development of a clinical-scale separator for the isolation of clinically relevant cells. For that application, we chose to employ a unique class of magnetic nanoparticles referred to as ferrofluids (Rosensweig, R. E., Ferrohydrodynamics, Cambridge University Press, New York, 1985). These materials possess many unique properties that can be exploited in biological applications, and we have extensive experience successfully employing these materials in a variety of separation-related applications (U.S. Pat. Nos. 5,200,084 A; 5,466,574 A; 5,622,831 A; 5,698,271 A; 5,876,593 A; 6,551,843 B1; 6,623,982 B1; 6,645,731 B2; and 7,332,288 B2, each of which is hereby incorporated by reference in its entirety). Our aqueous ferrofluids are stable colloids comprising magnetic cores (ca. 100 nm) of quasi-spherical clusters of magnetite crystals coated with multilayers of human serum albumin (HSA). These so-called HSA-ferrofluids are made by modifications of methods disclosed by Liberti, et al. (U.S. Pat. No. 6,120,856, which is hereby incorporated by reference in its entirety), resulting in materials with greater stability and lower non-specific binding. Various common-capture agents (e.g., anti-mouse IgG or streptavidin) can be covalently coupled, resulting in particles with an average hydrodynamic size from about 100 to about 200 nm. These so-called common-capture ferrofluids, which contain the particles, have several unique characteristics: 1) despite their small size, they are extraordinarily magnetic, allowing labeled targets to be separated with relatively simple magnetic separators composed of permanent magnets (as opposed to approaches to generate high-gradient magnetic fields, such as placing columns packed with steel wool or ferromagnetic beads within an external magnetic field); 2) they are non-toxic to cells; 3) their protein coating renders them biocompatible with cells and confers low non-specific binding to non-target cells; 4) they are colloidal, allowing them to bind cells with reaction-diffusion kinetics, thus incubations times are short and agitation is not required; 5) their manufacture is relatively straightforward; and 6) they are readily filter-sterilized.

The present embodiments were prepared in a manner to increase efficiency and to reduce the amount of time and steps to activate and separate cells from a sample, such as a blood product. Accordingly, the methods provided herein facilitate simultaneous separation and activation of T cells, or subsets thereof, and allow for simple subsequent stimulation with co-stimulatory agents or a combination of co-stimulatory agents at a desired level or time point following separation and activation. Purified T cells or T cell subsets that have been activated and stimulated in this manner can be genetically modified and expanded to sufficient numbers for use in therapy using other methods known to a person skilled in the art.

In contrast to other activation methods currently in use, the disclosed methods obviate the need to first perform one or more T cell isolations, as separation and activation are performed simultaneously. Accordingly, in some embodiments, the separation and activation occur simultaneously. As a consequence, in some embodiments, one performing the methods described herein can use a more complex mixture of cells (e.g., a leukapheresis product) than is allowed using other activation methods, which has significant advantages in throughput and cost-reduction. Moreover, the ability to activate and separate at the same time allows T cells, or subsets thereof, to be isolated using either positive or negative selection. Since stimulation is accomplished by simply adding a soluble co-stimulatory agent or combination of soluble co-stimulatory agents at any desired level or time point following separation and activation, the methods are adaptable to different workflows and give the practitioner a high degree of flexibility to tailor the stimulatory signal(s) as appropriate to the specific sample. In comparison to large particles typically used for T cell activation, the particles employed herein (as well as the soluble agents) can be filter-sterilized and need not be removed for downstream processing as they are biocompatible, non-toxic, and do not interfere with expansion or genetic modification.

Thus, provided herein are methods of labeling cells in blood products such that they can be simultaneously separated and activated, with subsequent stimulation to expand T cells by adding one or more soluble co-stimulatory agents. The embodiments are described in more detail herein. Each of the embodiments described herein can be combined with one another in any manner as would be evident from the present disclosure.

Embodiments described herein provide for methods of simultaneously separating and activating a population of T cells, or subsets thereof, the method comprising incubating a sample comprising a population of labeled magnetic particles, with at least one antibody that binds to a T cell surface protein and activates the T cell, and a blood product; applying a magnetic force to the sample; and separating the cells that are bound to the magnetic particles from the cells that are not bound to the magnetic particles, wherein the labeled magnetic particles are labeled with a common-capture reagent. In some embodiments, the labeled magnetic particles and the at least one antibody are mixed prior to being mixed with the blood product. In some embodiments, the at least one antibody and the blood product are mixed prior to being mixed with the labeled magnetic particles. In some embodiments, the labeled magnetic particles and the blood product are mixed prior to being mixed with the at least one antibody.

In some, or all, of the embodiments described herein, the cells that are captured for expansion are cultured in the presence of a co-stimulatory agent. The co-stimulatory agent can be soluble. In some embodiments, the co-stimulatory agent is not bound to a particle as described herein. The cells can be cultured while being bound to the magnetic or non-magnetic particles or can be released from the magnetic particles or non-magnetic particles after the separation step.

In some or all of the embodiments described herein, once the cells are separated and activated, the cells can be cultured in the presence of soluble co-stimulatory agent. In some embodiments, the co-stimulatory agent is anti-CD28 antibody, B7-1, B7-2, anti-CD2, and/or LFA-3. This is a non-limiting list of exemplary co-stimulatory agents and other agents can be used to stimulate the expansion and growth of the separated and activated T cells. In some embodiments, the co-stimulatory agent is soluble. In some embodiments, the co-stimulatory agent is not bound to a particle, which can also be referred to as a bead. In some embodiments, the cells are cultured in one or more of the co-stimulatory agents. In some embodiments, the co-stimulatory agent is mouse-derived anti-human CD28 of the IgG1 subclass. In some embodiments, the soluble co-stimulatory agent is a mixture of a mouse-derived anti-human CD28 of the IgG1 subclass and a mouse-derived anti-human CD2 of the IgG1 subclass. In some embodiments, the co-stimulatory agent is added from about 1 minute to about 20 hours after the separating step. In some embodiments, the co-stimulatory agent is added from about 2 minutes to about 10 hours after the separating step. The co-stimulatory agent can be added at multiple time points during that period. In some embodiments, the co-stimulatory agent is added at a single time point after the separating step, but no more than about 20 hours after the separating step. In some embodiments, the co-stimulatory agent is added immediately after the separating step. In some embodiments, the co-stimulatory agent is added about 1 minute to about 20 hours after the separating step. In some embodiments, the co-stimulatory agent is added at different time points after the separating step, wherein the co-stimulatory agent is not added more than about 20 hours after the separating step. As discussed herein, the cells can be cultured in the presence of a co-stimulatory agent. Because the addition of the co-stimulatory agent can be decoupled from the simultaneous separation and activation step, the level of the soluble co-stimulatory agent can be independently varied with respect to the level of the activation antibody. Moreover, the timing can be user-defined to some extent as well, although an upper limit exists to prevent cell anergy. In some embodiments, the level of the at least one soluble co-stimulatory agent is 20-fold higher than the level of the activation antibody. Wherein multiple soluble co-stimulatory agents are employed, this method allows for their control with respect to one another and with respect to the level of the activation antibody. The timing can also be varied here as well. In some embodiments, multiple soluble co-stimulatory agents are added at one time point from about 1 minute to about 20 hours after the separating step, or as otherwise described herein. In some embodiments, multiple soluble co-stimulatory agents are added at different time points from about 1 minute to about 20 hours after the separating step, or as otherwise described herein.

The particles described herein for any of the embodiments described herein can be any size. In some embodiments, the particles have an average size of about 50 nm to about 200 nm, about 50 nm to about 150 nm, about 75 to about 150 nm, about 100 to about 200 nm, about 50 nm to about 150 nm, or about 75 to about 250 nm. In some embodiments, the size of the particle is about 130 nm to about 150 nm. The particles can be magnetic or non-magnetic as described herein. The exact composition of the non-magnetic particle is not critical; however, it could be biodegradable or degradable in response to a stimulus, and it can be biocompatible, non-toxic, inert and similar in size to the magnetic nanoparticle. In some embodiments, the non-magnetic nanoparticle is comprised of latex. In some embodiments, the non-magnetic nanoparticle is silica. In some embodiments, the non-magnetic nanoparticle is comprised of biodegradable poly(lactic-co-glycolic acid).

As used herein the blood product is a whole peripheral blood product, a leukapheresis product, or a buffy coat blood product. In some embodiments, the blood product comprises mononuclear cells obtained from peripheral blood. In some embodiments, the blood product comprises a population of enriched T cells. In some embodiments, blood product comprises at least one population of an enriched T cell subset. In some embodiments, the blood product is not a purified blood product. A blood product that is not a purified blood product can be blood that is taken from a donor that is not filtered or otherwise purified after being taken from the donor.

In some embodiments, the incubation of the blood product, the magnetic particles, and the at least one antibody that binds to a T cell surface protein and activates the T cell is about 5 to about 30 minutes. In some embodiments, the incubation is about 10 minutes.

In some embodiments, a magnetic force is applied for a total of about 5 to about 30 minutes in order to achieve a magnetic separation. This can be referred to as the magnetic separation step. In some embodiments, the magnetic force is applied for a total of about 10 minutes to separate the magnetic particles, and the cells that are bound to the same, from the solution. In some embodiments, the magnetic force is applied for a total of about 15 minutes to separate the magnetic particles, and the cells that are bound to the same, from the solution. This force can be applied as a constant force to ensure maximal separation.

Additionally, in some embodiments, prior to a constant magnetic force being applied to separate the magnetic particles from the solution, a magnetic force can be applied in cycles to promote nanoparticle-cell interaction. For example, after the blood product, the magnetic particles, and the least one antibody are incubated together for a period of time, the mixture is exposed to a magnetic force for a period of time and then agitated to redistribute the particles. Without being bound to any particular theory, the cycles can increase the interactions between the particles and the cells, which should increase the binding of the cells and the particles. This is an alternative manner in which to mix the components in solution. The cycle can then be repeated. In some embodiments, these cycles can be repeated 1, 2, 3, 4, 5, 6, 7, 8, 9, 10, 20, 30, 40, 50, or 60 times. In some embodiments, this cycle is repeated at least 1, 2, 3, 4, 5, 6, 7, 8, 9, 10, 20, 30, 40, 50, or 60 times. In some embodiments, about 5 to about 100 cycles, about 10 to about 90 cycles, about 20 to about 60 cycles, about 30 to about 60 cycles, about 40 to about 80 cycles, or about 50 to about 100 cycles are performed. In some embodiments, the cycles are performed for about 10 seconds each for about 10 minutes. In some embodiments, the cycles are performed for about 20 seconds each for about 10 minutes. In some embodiments, the cycles are performed for about 30 seconds each for about 10 minutes. In some embodiments, the magnetic force is applied as an intermittent magnetic field gradient during the incubation step. In some embodiments, the magnetic force is applied as an intermittent magnetic field gradient during the incubation step for about 10 seconds to about 30 seconds. The embodiments described herein in relation to the application of the magnetic force can be applied to any of the methods described herein. In some embodiments, the mixture is subjected to intermittent magnetic field gradients via cycles of exposure to a magnetic field gradient with subsequent brief agitation. Thus, for example, the magnetic nanoparticles can be moved relative to the other elements in the mixture, which can promote cell labeling. Without being bound to any particular theory, the magnetic field gradient is able to polarize magnetic nanoparticles on the cell surface, which can promote receptor ligation. In some embodiments, the mixture of the antibody, common-capture particles, and cells is statically incubated. In some embodiments, the mixture of the antibody, common-capture magnetic particles, and cells is subjected to intermittent magnetic field gradients via cycles of exposure to a magnetic gradient with subsequent brief agitation. In some embodiments, the duration of each exposure to a magnetic gradient is from about 5 seconds to about 60 seconds. This can be done for cycles for about 5 to about 15 minutes. In some embodiments, the duration of each exposure to a magnetic gradient is about 10 seconds for a total time of about 10 minutes. In some embodiments, the duration of each exposure to a magnetic gradient is about 30 seconds for a total time of about 10 minutes. Although this section about applying a magnetic field in cycles may be presented in proximity to these embodiments, these embodiments of applying a magnetic field in cycles can be used in any of the methods described herein.

In some embodiments, the at least one antibody that binds to a T cell surface protein and activates the T cell binds to the magnetic particle. In some embodiments, the at least one antibody binds to the common-capture reagent bound to the magnetic particle. In some embodiments, the common-capture reagent is an anti-IgG antibody as described herein. In some embodiments, the anti-IgG antibody is an anti-IgG1 subclass antibody. In some embodiments, the at least one first antibody is labeled with biotin or a derivative thereof (e.g. biotinylated) and the common-capture reagent is a reagent that binds to biotin or a derivative thereof. Examples of such reagents include, but are not limited to, streptavidin, native avidin, deglycosylated avidin, anti-biotin antibody, and combinations thereof.

In some embodiments, the at least one antibody is an activating antibody. An activating antibody is an antibody that can bind to a T cell and activate it. Examples of such antibodies include, but are not limited to anti-CD3 antibodies and/or anti-CD2 antibodies and the like. In some embodiments, the at least one antibody or activating antibody is an anti-CD3 antibody. In some embodiments the anti-CD3 antibody is an anti-human CD3 antibody.

Embodiments provided herein also provide for the simultaneous separation and activation of a population of T cells, or subsets thereof, by separating and activating cells that are not bound to magnetic particles. In some embodiments, the methods comprise: a) incubating a sample comprising: a blood product; a population of non-magnetic particles bound to at least one first antibody that binds to a T cell surface protein and activates a T cell in the blood product; a population of magnetic particles bound to at least one second antibody that binds to a cell surface protein of a cell in the blood product that is not a T cell or not a T cell of the desired sub-population, wherein the second antibody does not bind to the non-magnetic particles; b) applying a magnetic force to the sample; c) separating the cells that are bound to the magnetic particles from the cells that are not bound to the magnetic particles; and d) optionally culturing the cells that are not bound to the magnetic particles to expand the population of cells. Without being bound to any particular theory, these embodiments allow for the activation of all T cells in the blood product, but only separating those of a desired sub-population of T cells. This can be done, for example, by activating the entire population by incubation with an anti-CD3 antibody. Thus, in some embodiments, the first antibody is an anti-CD3 antibody. In some embodiments, the first antibody is anti-human CD3 antibody, or fragments thereof, labeled with biotin, or a derivative thereof. If the first antibody is labeled with biotin, or a derivative thereof, then the magnetic particles are labeled with a reagent that binds to biotin or a derivative thereof. The first antibody can also be differentiated from the second antibody based upon the species of antibody being used. Then the magnetic particles can be bound with anti-IgG antibody that is species-specific and will not bind to the second antibody. For example, if the first antibody is produced in a rat and the second antibody is produced in a sheep, then the magnetic particles can be coated with a rat anti-IgG antibody that will bind to the first antibody, but not to the second antibody. This will allow the different particles to bind to different antibodies. In some embodiments, the first antibody is an anti-CD3 and/or an anti-CD2 antibody.

In some embodiments, the first antibody is anti-human CD3 antibody, or fragments thereof, labeled with biotin, or a derivative thereof, wherein the first antibody is not a mouse anti-human CD3 antibody of the IgG1 subclass. In such embodiments, the non-magnetic particle is labeled with a common-capture reagent that binds to biotin or a derivative thereof. In some embodiments, the common-capture reagent is streptavidin, native avidin, deglycosylated avidin, or anti-biotin antibody, or combinations thereof. In some embodiments, the first antibody is an anti-human CD3 antibody and binds to anti-IgG antibody on the first non-magnetic particle, but does not bind to the magnetic particle.

In some embodiments, the at least one second antibody can be referred to as a separation antibody. That is, the antibodies are used to separate non-desired cells out of the desired cell population. These separation antibodies can be specific for non-T cells. In some embodiments, the at least one second antibody, or separation antibody, is one or more of anti-CD11b, anti-CD16, anti-CD19, anti-CD36, anti-CD41a, anti-CD56, or anti-CD235a antibodies. In some embodiments, the separation antibody can also be referred to as the first antibody. Whether or not an antibody or a plurality of antibodies is referred to as a first or second antibody is not critical. Additionally, this list of antibodies is non-limiting and any other antibody could be used to separate cells from a population being processed based upon the preference of the user. The separation antibody can be chosen based upon the desired purpose. Although listed here, these separation antibodies can be used in conjunction with any of the embodiments described herein. Here, the magnetic particles are separating the non-desired cells, those that are not being activated and expanded. Thus, the magnetic force is separating away the non-desired cells and the cells left over, thus negatively selected, are the cells that are being activated and expanded when they are cultured as described herein.

In some embodiments, a magnetic force is applied for a total of about 5 to about 30 minutes in order to promote nanoparticle-cell interaction. This can be referred to as the magnetic separation step. In some embodiments, the magnetic force is applied for a total of about 10 minutes to separate the magnetic particles, and the cells that are bound to the same, from the solution. In some embodiments, the magnetic force is applied for a total of about 15 minutes to separate the magnetic particles, and the cells that are bound to the same, from the solution. This force can be applied as a constant force to ensure maximal separation.

Additionally, in some embodiments, prior to a constant magnetic force being applied to separate the magnetic particles from the solution, a magnetic force can be applied in cycles to promote nanoparticle-cell interaction. For example, after the blood product, the magnetic particles, and the least one antibody are incubated together for a period of time, the mixture is exposed to a magnetic force for a period of time and then agitated to redistribute the particles. Without being bound to any particular theory, the cycles can increase the interactions between the particles and the cells, which should increase the binding of the cells and the particles. This is an alternative manner in which to mix the components in solution. The cycle can then be repeated. In some embodiments, these cycles can be repeated 1, 2, 3, 4, 5, 6, 7, 8, 9, 10, 20, 30, 40, 50, or 60 times. In some embodiments, this cycle is repeated at least 1, 2, 3, 4, 5, 6, 7, 8, 9, 10, 20, 30, 40, 50, or 60 times. In some embodiments, about 5 to about 100 cycles, about 10 to about 90 cycles, about 20 to about 60 cycles, about 30 to about 60 cycles, about 40 to about 80 cycles, or about 50 to about 100 cycles are performed. In some embodiments, the cycles are performed for about 10 seconds each for about 10 minutes. In some embodiments, the cycles are performed for about 20 seconds each for about 10 minutes. In some embodiments, the cycles are performed for about 30 seconds each for about 10 minutes. In some embodiments, the magnetic force is applied as an intermittent magnetic field gradient during the incubation step. In some embodiments, the magnetic force is applied as an intermittent magnetic field gradient during the incubation step for about 10 seconds to about 30 seconds. The embodiments described herein in relation to the application of the magnetic force can be applied to any of the methods described herein. In some embodiments, the mixture is subjected to intermittent magnetic field gradients via cycles of exposure to a magnetic field gradient with subsequent brief agitation. Thus, for example, the magnetic nanoparticles can be moved relative to the other elements in the mixture, which can promote cell labeling. Without being bound to any particular theory, the magnetic field gradient is able to polarize magnetic nanoparticles on the cell surface, which can promote receptor ligation. In some embodiments, the mixture of the antibody, common-capture particles, and cells is statically incubated. In some embodiments, the mixture of the antibody, common-capture magnetic particles, and cells is subjected to intermittent magnetic field gradients via cycles of exposure to a magnetic gradient with subsequent brief agitation. In some embodiments, the duration of each exposure to a magnetic gradient is from about 5 seconds to about 60 seconds. This can be done for cycles for about 5 to about 15 minutes. In some embodiments, the duration of each exposure to a magnetic gradient is about 10 seconds for a total time of about 10 minutes. In some embodiments, the duration of each exposure to a magnetic gradient is about 30 seconds for a total time of about 10 minutes. Although this section about applying a magnetic field in cycles may be presented in proximity to these embodiments, these embodiments of applying a magnetic field in cycles can be used in any of the methods described herein.

As for other embodiments, once the cells are separated and activated, the cells can be cultured in the presence of soluble co-stimulatory agent. In some embodiments, the co-stimulatory agent is anti-CD28 antibody, B7-1, B7-2, anti-CD2, and/or LFA-3. In some embodiments, the co-stimulatory agent is soluble. In some embodiments, the co-stimulatory agent is not bound to a particle, which can also be referred to as a bead. In some embodiments, the cells are cultured in one or more of the co-stimulatory agents. In some embodiments, the co-stimulatory agent is mouse-derived anti-human CD28 of the IgG1 subclass. In some embodiments, the soluble co-stimulatory agent is a mixture of a mouse-derived anti-human CD28 of the IgG1 subclass and a mouse-derived anti-human CD2 of the IgG1 subclass. In some embodiments, the co-stimulatory agent is added from about 1 minute to about 20 hours after the separating step. In some embodiments, the co-stimulatory agent is added from about 2 minutes to about 10 hours after the separating step. The co-stimulatory agent can be added at multiple time points during that period. In some embodiments, the co-stimulatory agent is added at a single time point after the separating step, but no more than about 20 hours after the separating step. In some embodiments, the co-stimulatory agent is added immediately after the separating step. In some embodiments, the co-stimulatory agent is added about 1 minute to about 20 hours after the separating step. In some embodiments, the co-stimulatory agent is added at different time points after the separating step, wherein the co-stimulatory agent is not added more than about 20 hours after the separating step. As discussed herein, the cells can be cultured in the presence of a co-stimulatory agent. Because the addition of the co-stimulatory agent can be decoupled from the simultaneous separation and activation step, the level of the soluble co-stimulatory agent can be independently varied with respect to the level of the activation antibody. Moreover, the timing can be user-defined to some extent as well, although an upper limit exists to prevent cell anergy. In some embodiments, the level of the at least one soluble co-stimulatory agent is 20-fold higher than the level of the activation antibody. Wherein multiple soluble co-stimulatory agents are employed, this method allows for their control with respect to one another and with respect to the level of the activation antibody. The timing can also be varied here as well. In some embodiments, multiple soluble co-stimulatory agents are added at one time point from about 1 minute to about 20 hours after the separating step, or as otherwise described herein. In some embodiments, multiple soluble co-stimulatory agents are added at different time points from about 1 minute to about 20 hours after the separating step, or as otherwise described herein.

In some embodiments, methods of simultaneously separating and activating a sub-population of T cells, or subsets thereof are provided, the method comprising:

a) incubating a sample comprising:
a blood product;
a population of magnetic particles bound to at least one first antibody that binds to a cell surface protein of a desired sub-populations of cells in the blood product;
a population of non-magnetic particles bound to at least one second antibody that binds to and activates the desired sub-population of cells in the blood product, wherein the second antibody does not bind to the magnetic particles;

b) applying a magnetic force to the sample;

c) separating the cells that are bound to the magnetic particles from the cells that are not bound to the magnetic particles; and

d) optionally culturing the cells that are bound to the magnetic particles to expand the sub-population of the cells.

In some embodiments, the first antibody is an anti-CD4 antibody or anti-CD8 antibody. In some embodiments, the second antibody is an anti-CD3 antibody and/or anti-CD2 antibody. In some embodiments, the first antibody binds to the magnetic particles through a common-capture reagent with which the magnetic particles are labeled. In some embodiments, the common-capture reagent is an anti-IgG antibody. In some embodiments, the common-capture reagent is a reagent that binds to biotin, or a derivative thereof. Examples of such common-capture reagents are described herein. If the common-capture reagent on the magnetic particles is a reagent that binds to biotin, or a derivative thereof, then the first antibody is a biotinylated antibody. The common-capture reagent on the magnetic particles should be different from any common-capture reagent on the second particle. For example, if the common-capture reagent on the magnetic particles is a reagent that binds to biotin, or a derivative thereof, the non-magnetic particles does not have a common-capture reagent that binds to biotin, or a derivative thereof, or vice versa. Another example, is that if the common-capture reagent binds to antibodies that are from rats, such as rat anti-IgG, then the other common-capture reagent will not bind to the same type of antibody. Thus, this ensures that the magnetic particles and the non-magnetic particles do not bind to the same antibodies. For the avoidance of doubt, it is understood that antibodies are not always 100% specific for the target. Thus, the cells through their surface proteins that bind to the different antibodies may be present in both populations of particles, but the different populations will be enriched for the population of cells that are intended to react with the antibody.

In some embodiments, a magnetic force is applied for a total of about 5 to about 30 minutes in order to promote nanoparticle-cell interaction. This can be referred to as the magnetic separation step. In some embodiments, the magnetic force is applied for a total of about 10 minutes to separate the magnetic particles, and the cells that are bound to the same, from the solution. In some embodiments, the magnetic force is applied for a total of about 15 minutes to separate the magnetic particles, and the cells that are bound to the same, from the solution. This force can be applied as a constant force to ensure maximal separation.

Additionally, in some embodiments, prior to a constant magnetic force being applied to separate the magnetic particles from the solution, a magnetic force can be applied in cycles to promote nanoparticle-cell interaction. For example, after the blood product, the magnetic particles, and the least one antibody are incubated together for a period of time, the mixture is exposed to a magnetic force for a period of time and then agitated to redistribute the particles. Without being bound to any particular theory, the cycles can increase the interactions between the particles and the cells, which should increase the binding of the cells and the particles. This is an alternative manner in which to mix the components in solution. The cycle can then be repeated. In some embodiments, these cycles can be repeated 1, 2, 3, 4, 5, 6, 7, 8, 9, 10, 20, 30, 40, 50, or 60 times. In some embodiments, this cycle is repeated at least 1, 2, 3, 4, 5, 6, 7, 8, 9, 10, 20, 30, 40, 50, or 60 times. In some embodiments, about 5 to about 100 cycles, about 10 to about 90 cycles, about 20 to about 60 cycles, about 30 to about 60 cycles, about 40 to about 80 cycles, or about 50 to about 100 cycles are performed. In some embodiments, the cycles are performed for about 10 seconds each for about 10 minutes. In some embodiments, the cycles are performed for about 20 seconds each for about 10 minutes. In some embodiments, the cycles are performed for about 30 seconds each for about 10 minutes. In some embodiments, the magnetic force is applied as an intermittent magnetic field gradient during the incubation step. In some embodiments, the magnetic force is applied as an intermittent magnetic field gradient during the incubation step for about 10 seconds to about 30 seconds. The embodiments described herein in relation to the application of the magnetic force can be applied to any of the methods described herein. In some embodiments, the mixture is subjected to intermittent magnetic field gradients via cycles of exposure to a magnetic field gradient with subsequent brief agitation. Thus, for example, the magnetic nanoparticles can be moved relative to the other elements in the mixture, which can promote cell labeling. Without being bound to any particular theory, the magnetic field gradient is able to polarize magnetic nanoparticles on the cell surface, which can promote receptor ligation. In some embodiments, the mixture of the antibody, common-capture particles, and cells is statically incubated. In some embodiments, the mixture of the antibody, common-capture magnetic particles, and cells is subjected to intermittent magnetic field gradients via cycles of exposure to a magnetic gradient with subsequent brief agitation. In some embodiments, the duration of each exposure to a magnetic gradient is from about 5 seconds to about 60 seconds. This can be done for cycles for about 5 to about 15 minutes. In some embodiments, the duration of each exposure to a magnetic gradient is about 10 seconds for a total time of about 10 minutes. In some embodiments, the duration of each exposure to a magnetic gradient is about 30 seconds for a total time of about 10 minutes. Although this section about applying a magnetic field in cycles may be presented in proximity to these embodiments, these embodiments of applying a magnetic field in cycles can be used in any of the methods described herein.

As for other embodiments described herein, once the cells are separated and activated, the cells can be cultured in the presence of soluble co-stimulatory agent. In some embodiments, the co-stimulatory agent is anti-CD28 antibody, B7-1, B7-2, anti-CD2, and/or LFA-3. In some embodiments, the co-stimulatory agent is soluble. In some embodiments, the co-stimulatory agent is not bound to a particle, which can also be referred to as a bead. In some embodiments, the cells are cultured in one or more of the co-stimulatory agents. In some embodiments, the co-stimulatory agent is mouse-derived anti-human CD28 of the IgG1 subclass. In some embodiments, the soluble co-stimulatory agent is a mixture of a mouse-derived anti-human CD28 of the IgG1 subclass and a mouse-derived anti-human CD2 of the IgG1 subclass. In some embodiments, the co-stimulatory agent is added from about 1 minute to about 20 hours after the separating step. In some embodiments, the co-stimulatory agent is added from about 2 minutes to about 10 hours after the separating step. The co-stimulatory agent can be added at multiple time points during that period. In some embodiments, the co-stimulatory agent is added at a single time point after the separating step, but no more than about 20 hours after the separating step. In some embodiments, the co-stimulatory agent is added immediately after the separating step. In some embodiments, the co-stimulatory agent is added about 1 minute to about 20 hours after the separating step. In some embodiments, the co-stimulatory agent is added at different time points after the separating step, wherein the co-stimulatory agent is not added more than about 20 hours after the separating step. As discussed herein, the cells can be cultured in the presence of a co-stimulatory agent. Because the addition of the co-stimulatory agent can be decoupled from the simultaneous separation and activation step, the level of the soluble co-stimulatory agent can be independently varied with respect to the level of the activation antibody. Moreover, the timing can be user-defined to some extent as well, although an upper limit exists to prevent cell anergy. In some embodiments, the level of the at least one soluble co-stimulatory agent is 20-fold higher than the level of the activation antibody. Wherein multiple soluble co-stimulatory agents are employed, this method allows for their control with respect to one another and with respect to the level of the activation antibody. The timing can also be varied here as well. In some embodiments, multiple soluble co-stimulatory agents are added at one time point from about 1 minute to about 20 hours after the separating step, or as otherwise described herein. In some embodiments, multiple soluble co-stimulatory agents are added at different time points from about 1 minute to about 20 hours after the separating step, or as otherwise described herein.

Embodiments provided herein also provide methods of simultaneously separating and activating of a sub-population of T cells, or subsets thereof, the method comprising:

a) incubating a sample comprising:
a blood product;
a population of magnetic particles bound to at least one first antibody that binds to the cells not in a desired sub-populations of T cells in the blood product; and
a population of non-magnetic particles bound to at least one second antibody that binds to and activates the desired sub-populations of T cells in the blood product, wherein the second antibody does not bind to the magnetic particles;

b) applying a magnetic force to the sample;

c) separating the cells that are bound to the magnetic particles from the cells that are not bound to the magnetic particles; and

d) optionally culturing the cells that are not bound to the magnetic particles to expand the sub-population of the cells.

In some embodiments, the first antibody does not bind to the non-magnetic particles.

The at least one first antibody can be a plurality of antibodies that bind to the cells that are desired to be separated away from the cells that are intended to be activated and expanded according to the methods described herein. Thus, the at least one first antibody can be have multiple antibodies that are specific for different cell types, except that the at least one first antibody would not comprise an antibody that binds to the cells that would be cultured after being activated. For example, in some embodiments, the at least one first antibody is chosen from the group comprising anti-CD11b, anti-CD16, anti-CD19, anti-CD36, anti-CD41a, anti-CD56, anti-CD235a, anti-CD4, and anti-CD8 antibody, or any combination thereof. However, in some embodiments, where it is desired that the CD4+ cells are being captured, but not the CD8+ cells, the at least one first antibody would not comprise an anti-CD4 antibody, which would allow the CD4+ cells to be separated and activated away from the blood product. However, the at least one first antibody would have an anti-CD8 antibody. In some embodiments, if one desired to collect the CD8+ cells, but not the CD4+ cells, then the at least one first antibody would not comprise an anti-CD8 antibody, but would have an anti-CD4 antibody. The lists of antibodies are for example purposes only and any desired antibody could be used to select the cells that are being captured by the magnetic particles.

In some embodiments, the at least one second antibody is biotinylated. In some embodiments, the at least one first antibody is not biotinylated. In some embodiments, the second antibody is an anti-CD3 and/or anti-CD2 antibody. These antibodies can bind to the cells that are also being captured by the magnetic particles. However, any cells that are also being bound to the magnetic particles will be separated from the cells that are only bound to the non-magnetic particles during the separation step where the magnetic force is applied as described herein. In some embodiments, the antibody that activates the cell can be a combination of antibodies that can activate T cells, regardless of the population being separated and activated. As discussed herein, in other embodiments, the magnetic and non-magnetic particles can be coated or labeled with common-capture reagents, but not with the same ones to prevent or limit cross-reactivity. Thus, if one particle population is coated with a common-capture reagent that binds to biotin, or a derivative thereof, such that it can bind to a biotinylated antibody, the other particle population is not coated with a similar common-capture reagent. In another example, the common-capture reagent is an anti-IgG antibody. In such embodiments, the other common-capture reagent is either not an anti-IgG antibody or is an anti-IgG antibody that does not recognize the same species or subclass of antibody. When referring to a common-capture reagent recognizing a species of antibody, it is referring to the species in which the antibody was produced, not the species of the protein that is recognized by the specific antibody.

Additionally, in some embodiments, prior to a constant magnetic force being applied to separate the magnetic particles from the solution, a magnetic force can be applied in cycles to promote nanoparticle-cell interaction. For example, after the blood product, the magnetic particles, and the least one antibody are incubated together for a period of time, the mixture is exposed to a magnetic force for a period of time and then agitated to redistribute the particles. Without being bound to any particular theory, the cycles can increase the interactions between the particles and the cells, which should increase the binding of the cells and the particles. This is an alternative manner in which to mix the components in solution. The cycle can then be repeated. In some embodiments, these cycles can be repeated 1, 2, 3, 4, 5, 6, 7, 8, 9, 10, 20, 30, 40, 50, or 60 times. In some embodiments, this cycle is repeated at least 1, 2, 3, 4, 5, 6, 7, 8, 9, 10, 20, 30, 40, 50, or 60 times. In some embodiments, about 5 to about 100 cycles, about 10 to about 90 cycles, about 20 to about 60 cycles, about 30 to about 60 cycles, about 40 to about 80 cycles, or about 50 to about 100 cycles are performed. In some embodiments, the cycles are performed for about 10 seconds each for about 10 minutes. In some embodiments, the cycles are performed for about 20 seconds each for about 10 minutes. In some embodiments, the cycles are performed for about 30 seconds each for about 10 minutes. In some embodiments, the magnetic force is applied as an intermittent magnetic field gradient during the incubation step. In some embodiments, the magnetic force is applied as an intermittent magnetic field gradient during the incubation step for about 10 seconds to about 30 seconds. The embodiments described herein in relation to the application of the magnetic force can be applied to any of the methods described herein. In some embodiments, the mixture is subjected to intermittent magnetic field gradients via cycles of exposure to a magnetic field gradient with subsequent brief agitation. Thus, for example, the magnetic nanoparticles can be moved relative to the other elements in the mixture, which can promote cell labeling. Without being bound to any particular theory, the magnetic field gradient is able to polarize magnetic nanoparticles on the cell surface, which can promote receptor ligation. In some embodiments, the mixture of the antibody, common-capture particles, and cells is statically incubated. In some embodiments, the mixture of the antibody, common-capture magnetic particles, and cells is subjected to intermittent magnetic field gradients via cycles of exposure to a magnetic gradient with subsequent brief agitation. In some embodiments, the duration of each exposure to a magnetic gradient is from about 5 seconds to about 60 seconds. This can be done for cycles for about 5 to about 15 minutes. In some embodiments, the duration of each exposure to a magnetic gradient is about 10 seconds for a total time of about 10 minutes. In some embodiments, the duration of each exposure to a magnetic gradient is about 30 seconds for a total time of about 10 minutes. Although this section about applying a magnetic field in cycles may be presented in proximity to these embodiments, these embodiments of applying a magnetic field in cycles can be used in any of the methods described herein.

As for other embodiments, once the cells are separated and activated, the cells can be cultured in the presence of soluble co-stimulatory agent. In some embodiments, the co-stimulatory agent is anti-CD28 antibody, B7-1, B7-2, anti-CD2, and/or LFA-3. In some embodiments, the co-stimulatory agent is soluble. In some embodiments, the co-stimulatory agent is not bound to a particle, which can also be referred to as a bead. In some embodiments, the cells are cultured in one or more of the co-stimulatory agents. In some embodiments, the co-stimulatory agent is mouse-derived anti-human CD28 of the IgG1 subclass. In some embodiments, the soluble co-stimulatory agent is a mixture of a mouse-derived anti-human CD28 of the IgG1 subclass and a mouse-derived anti-human CD2 of the IgG1 subclass. In some embodiments, the co-stimulatory agent is added from about 1 minute to about 20 hours after the separating step. In some embodiments, the co-stimulatory agent is added from about 2 minutes to about 10 hours after the separating step. The co-stimulatory agent can be added at multiple time points during that period. In some embodiments, the co-stimulatory agent is added at a single time point after the separating step, but no more than about 20 hours after the separating step. In some embodiments, the co-stimulatory agent is added immediately after the separating step. In some embodiments, the co-stimulatory agent is added about 1 minute to about 20 hours after the separating step. In some embodiments, the co-stimulatory agent is added at different time points after the separating step, wherein the co-stimulatory agent is not added more than about 20 hours after the separating step. As discussed herein, the cells can be cultured in the presence of a co-stimulatory agent. Because the addition of the co-stimulatory agent can be decoupled from the simultaneous separation and activation step, the level of the soluble co-stimulatory agent can be independently varied with respect to the level of the activation antibody. Moreover, the timing can be user-defined to some extent as well, although an upper limit exists to prevent cell anergy. In some embodiments, the level of the at least one soluble co-stimulatory agent is 20-fold higher than the level of the activation antibody. Wherein multiple soluble co-stimulatory agents are employed, this method allows for their control with respect to one another and with respect to the level of the activation antibody. The timing can also be varied here as well. In some embodiments, multiple soluble co-stimulatory agents are added at one time point from about 1 minute to about 20 hours after the separating step, or as otherwise described herein. In some embodiments, multiple soluble co-stimulatory agents are added at different time points from about 1 minute to about 20 hours after the separating step, or as otherwise described herein.

Accordingly, the present embodiments disclosed herein provide for the positive and negative selection and activation of T cells or a sub-population thereof. The methods reduce the number of steps that are required as compared to previous methods and lead to greater activation and expansion as shown in the Examples provided herein. Without being bound to any particular theory, the discoveries that led to these embodiments were the result of extensive studies on methods to isolate T cells from blood products (e.g., leukapheresis products, buffy coats, and even whole blood) using indirect methods to magnetically label cells. The magnetic particles described herein can be in the form of highly magnetic colloidal nanoparticles, which can also be referred to as ferrofluids. These ferrofluids can conveniently be produced over a wide range of sizes while remaining colloidal and be used for magnetic cell separations in simple vessels with relatively simple magnetic separators composed of permanent magnets (as opposed to, for instance, column-based separators which generate high-gradient magnetic fields). Advantageously, these materials can be readily filter-sterilized.

In some embodiments, the ratio of the particles to cells is modified. In some embodiments, the ratio of the particles to the cell is greater than 200 particles per cell. In some embodiments, the ratio of the particles to the cells is about 30 to about 120 particles per cell. In some embodiments, the ratio of particles to the cells is from about 45 to about 90 particles per cell. In some embodiments, the ratio of the particles is 60 particles per cell. For the avoidance of doubt, the particles are coated/labeled with a common-capture reagent as described herein that interacts with the antibody that binds to the cell population that is chosen to be activated and/or separated from the remaining cells regardless of whether the cells being isolated binds to the magnetic particles or the non-magnetic particles.

In some embodiments, methods are provided for positively selecting for and simultaneously activating at least one T cell subset from a mixture of cells simply by adding a second common-capture nanoparticle and at least one separation antibody. The activated T cells can then be subsequently stimulated in the same manner as described above. In some embodiments, the simultaneous separation and activation of the desired T cell subset or subsets is performed by combining at least one separation antibody, an activation antibody, a magnetic nanoparticle coated with a first common-capture agent capable of binding the at least one separation antibody, and a non-magnetic nanoparticle coated with a second common-capture agent capable of binding the activation antibody; neither the first common-capture agent can bind the activation antibody, nor the second common-capture agent can bind the at least one separation antibody. This cocktail of antibodies and nanoparticles is then immediately combined with a mixture of cells containing the desired T cell subset or subsets (e.g., CD4+ cells, CD8+ cells, or CD4+ cells and CD8+ cells). Assuming an activation antibody of anti-CD3 is used, all CD3+ cells present in the mixture will be activated by the non-magnetic nanoparticle; however, only the desired T cell subset or subsets will be magnetically labeled (having bound both non-magnetic nanoparticles and magnetic nanoparticles). After an appropriate incubation period, a magnetic separation is performed to recover magnetically labeled cells as described herein.

In some embodiments, methods are provided for separating and activating a desired T cell subset or subsets, the mixture of cells containing a population of T cells to which this method is applied can be pure or impure. In some embodiments, the methods are applied to a population of enriched T cells or at least one population of enriched T cell subsets. In some embodiments, the methods are applied to mononuclear cells obtained from peripheral blood. In some embodiments, the methods are applied to a leukapheresis product. In some embodiments, the methods are applied to whole peripheral blood.

As described herein, some of the methods employ an antibody that acts as a separation antibody. In some embodiments, the separation antibody is selected from the group comprising mouse-derived anti-human CD4 of the IgG1 subclass, mouse-derived anti-human CD8 of the IgG1 subclass, and combinations thereof. In some embodiments, these antibodies are combined with magnetic nanoparticle that is a solid, HSA-coated ferrofluid nanoparticle, and the first common-capture agent immobilized on the magnetic nanoparticle is rat-derived anti-mouse IgG1. In such a combination, it can be combined with a second antibody that acts as an activation antibody, which can be a biotinylated mouse-derived anti-human CD3 of the IgG2a subclass and the second common-capture agent immobilized on the solid non-magnetic is streptavidin. The cocktail of antibodies and both types of nanoparticles are rapidly combined, mixed with cells containing the desired T cell subset or subsets, and a magnetic force is employed to separate magnetically labeled cells, which are then recovered.

In some embodiments, at least one separation antibody is selected from the group comprising biotinylated mouse-derived anti-human CD4 of the IgG2a subclass, biotinylated mouse-derived anti-human CD8 of the IgG2a subclass, or combinations thereof. As described herein, the separation antibody can be any antibody that one of skill in the art wants to use to separate cells from the sample that is being processed, such as a blood product. Other examples of separation antibodies include, but are not limited to, anti-CD11b, anti-CD16, anti-CD19, anti-CD36, anti-CD41a, anti-CD56, anti-CD235a, anti-CD4, and anti-CD8 antibodies. In some embodiments, the magnetic nanoparticle can be a solid, HSA-coated ferrofluid nanoparticle, and the first common-capture agent immobilized on the magnetic nanoparticle is streptavidin. In some embodiments, where there is both a separation and activation antibody, the activation antibody, can be an anti-CD3 antibody. In some embodiments, the activation antibody is a mouse-derived anti-human CD3 of the IgG1 subclass and the second common-capture agent immobilized on the solid non-magnetic particle is rat-derived anti-mouse IgG1. In some embodiments, the cocktail of antibodies and both types of nanoparticles are rapidly combined, mixed with cells containing the desired T cell subset or subsets, and a magnetic force is employed to separate magnetically labeled cells, which are then recovered. As explained herein, this allows the different particles to bind to different cell populations.

As described herein, the methods can also be used to “negatively” select for and simultaneously activate T cells or at least one T cell subset from a mixture of cells. The activated T cells can then be subsequently stimulated in the same manner as described previously. For example, in some embodiments, the simultaneous negative selection and activation of T cells or at least one T cell subset (i.e., target cells) can be performed by combining at least one antibody that acts as a separation antibody capable of binding to non-target (not desired) cells, a second antibody that acts as an activation antibody, a magnetic nanoparticle coated with a first common-capture agent capable of binding the at least one separation antibody, and a non-magnetic nanoparticle coated with a second common-capture agent capable of binding the activation antibody, wherein neither the first common-capture agent can bind the activation antibody, nor the second common-capture agent can bind the at least one separation antibody. This cocktail of antibodies and nanoparticles is then immediately combined with a mixture of cells containing a population of T cells or the desired T cell subset(s) (e.g., CD4+ cells, CD8+ cells, or CD4+ cells and CD8+ cells). In some embodiments, for example, an activation antibody of anti-CD3 can be used, and all CD3+ cells present in the mixture will be activated by the non-magnetic nanoparticle. However, only the non-target cells that bound the at least one separation antibody will be magnetically labeled, while the T cells or desired T cell subset(s) will not be bound to the magnetic nanoparticle (having only bound the non-magnetic nanoparticles). After an appropriate incubation period, a magnetic separation is performed to remove magnetically labeled cells, which leaves the desired cells to be separated and further expanded in the presence or absence of an additional co-stimulatory agent as described herein.

As these methods can be used for both separation and activation of T cells or at least one T cell subset, the mixture of cells containing a population of T cells to which this method is applied can be pure or impure. In some embodiments, this method is applied to a population of enriched T cells or at least one population of enriched T cell subsets. In some embodiments, this method is applied to mononuclear cells obtained from peripheral blood. In yet another embodiment, this method is applied to a leukapheresis product. In still another embodiment, this method is applied to whole peripheral blood. Any other blood product described herein can also be used.

In some embodiments, the at least one separation antibody is any mouse-derived anti-human antibody of the IgG1 subclass that is capable of binding to non-target cells. Examples of such antibodies are provided herein. As explained herein, the separation antibody can be a single antibody or a plurality of antibodies. In some embodiments, the magnetic nanoparticle is a solid, HSA-coated ferrofluid nanoparticle, and the first common-capture agent immobilized on the magnetic nanoparticle is rat-derived anti-mouse IgG1, which will bind to the mouse-derived IgG1 subclass antibodies. In some embodiments, the activation antibody is biotinylated mouse-derived anti-human CD3 of the IgG2a subclass and the second common-capture agent immobilized on the solid non-magnetic particle is streptavidin. The cocktail of antibodies and both types of nanoparticles are rapidly combined, mixed with cells containing a population of T cells or the desired T cell subset(s), and a magnetic force is employed to separate magnetically labeled cells from non-magnetically labeled cells, the latter of which are then recovered. The second particles bind to the biotinylated antibodies that are bound to the target (desired) cells.

In some embodiments, at least one separation antibody is any biotinylated mouse-derived anti-human antibody of the IgG2a subclass that is capable of binding to non-target cells. The magnetic nanoparticle is a solid, HSA-coated ferrofluid nanoparticle, and the first common-capture agent immobilized on the magnetic nanoparticle is streptavidin. Further, the activation antibody is mouse-derived anti-human CD3 of the IgG1 subclass and the second common-capture agent immobilized on the solid non-magnetic particle is rat-derived anti-mouse IgG1. The cocktail of antibodies and both types of nanoparticles are rapidly combined, mixed with cells containing a population of T cells or the desired T cell subset(s), and a magnetic force is employed to separate magnetically labeled cells from non-magnetically labeled cells, the latter of which are then recovered.

The methods of the disclosed herein are distinct from prior methods, and they offer considerable utility and myriad advantages over other methods. For example, Table 1 provides a comparison of the methods disclosed herein to previous methods. In contrast to the commercially available methods, the methods disclosed herein, for example, do not require a pure T cell population, due to the unique approach wherein separation and activation are performed simultaneously. This translates into significant cost savings by substantially reducing processing time and effort, as well as the amount of reagents required. Concerning the latter, the embodiments disclosed herein are particularly conservative in terms of antibody and common-capture nanoparticles required to separate and activate T cells; as an example, to positively select and activate T cells from a leukapheresis product containing 5×10⁹ total nucleated cells, only 100 μg of anti-CD3 and 800 μg of common-capture ferrofluid is required. Unlike Dynabeads (ThermoFisher) and MACSiBeads (Miltenyi), the solid particles employed in the various methods described herein are small enough to allow for sterile filtration through a 0.22 μm filter, which is beneficial for its use in manufacturing a cellular product. Additionally, because in some embodiments, the stimulation is decoupled from the separation and activation methods, the relative amounts of activation and co-stimulation reagents can be varied, as can the timing of co-stimulation. Collectively, the differences that are highlighted between the methods disclosed herein and other methods illustrate that these methods provided significant and unexpected advantages as compared to other methods.

TABLE 1
Comparison of the methods disclosed herein to other methods.

| | Required | | Activator Size | Activator: | Timing |
|---|---|---|---|---|---|
| | Purity of | Simultaneous | & Format; 0.22 | Co-Stimulator | of Co- |
| | Cell | Separation & | μm Filter- | Levels | Stimulation |
| | Mixture | Activation? | Sterilizable? | Variable? | Variable? |
| Embodiments | Impure | Yes | 100-200 nm | Yes | Yes (0-16 h |
| Disclosed Herein | mixture or | | solid particle; | | after |
| | pure T cells | | Yes | | activation) |
| Dynabeads Human | Pure T cells | No | 4.5 μm solid | No | No |
| T-Activator | | | particle; No | | |
| CD3/CD28 | | | | | |
| (ThermoFisher) | | | | | |
| Anti-Biotin | Pure T cells | No | 3.5 μm solid | Yes | No |
| MACSiBeads | | | particle; No | | |
| (Miltenyi) | | | | | |
| TransAct T Cell | Pure T cells | No | 65 nm flexible | No | No |
| Reagent (Miltenyi) | | | nanomatrix; | | |
| | | | Yes | | |
| Immunocult | Pure T cells | No | Soluble TACs; | No | No |
| Human CD3/CD28 | | | Yes | | |
| T Cell Activator | | | | | |
| (STEMCELL) | | | | | |

The following examples are illustrative, but not limiting, of the methods and compositions described herein. Other suitable modifications and adaptations of the variety of conditions and parameters normally encountered in therapy and that are obvious to those skilled in the art are within the spirit and scope of the compounds and methods described herein.

### EXAMPLE 1

### Comparison of Activation and Expansion of T Cells Using Various Methods for Magnetic Labeling

To compare the activation and expansion of T cells using the various modified labeling methods, cells were positively selected and activated using three different modified labeling methods and stimulated through the addition of soluble anti-CD28 the following day. In all cases, PBMCs were isolated from human peripheral blood via the OptiPrep method (Axis Shield) by combining whole blood with 1.25 mL of OptiPrep per 10 mL of blood and centrifuging for 30 min at 1500 rcf (20° C.). The PBMC layer was removed and washed by centrifugation with Mg- and Ca-free DPBS (Sigma) containing 1% HSA to pellet the cells. The cell pellet was re-suspended and re-centrifuged at 300 rcf two more times to remove platelets.

In a first method, 6 μg/mL anti-CD3 antibody of the IgG1 subclass (Ancell) was combined with an equivalent volume of 48 μg/mL common-capture RAM-ferrofluid and vortexed. The PBMC were added to the above mixture in equal volumes at a final cell concentration of 3×10⁷ cells/mL (final [anti-CD3]=1.5 μg/mL; final [RAM-ferrofluid]=12 μg/mL), and the mixture was subjected to intermittent magnetic field gradients via cycles of exposure to a magnetic field gradient for 10 s with subsequent brief agitation for a total period of 10 min. The cell mixture was then placed in a quadrupole magnetic separator for 15 min. At the end of the separation, the non-magnetic cell fraction was removed via Pasteur pipette aspiration, and fresh buffer (DPBS, 1% HSA) was added to the tube without resuspension of the magnetically collected cells and incubated with the magnetic cell fraction for 10 min. This process was repeated once more. After the final aspiration of the non-magnetic cell fraction, the sample was removed from the magnetic field gradient and the magnetic cell fraction was re-suspended in ImmunoCult XF T-cell expansion medium (STEMCELL Technologies) supplemented with 100 IU/mL IL-2 (Gibco) to a total concentration of 1×10⁶ cells/mL and incubated at 37° C. (5% CO₂). Following an overnight incubation, 0.5 μg/mL mouse anti-human CD28 antibody of the IgG1 subclass (Mabtech) was added to the incubated cell fraction. The cells were periodically agitated and diluted to 1×10⁶ cells/mL with fresh expansion medium, and after 4 days in culture, the cells were analyzed for the presence of the CD25 marker via flow cytometry (FlowSight, Amnis). Upon analysis, 89% expressed the CD25 surface marker on Day 4, and cells experienced a 311-fold expansion by Day 15.

In a second method, 1.5 μg/mL anti-CD3 antibody of the IgG1 subclass (Ancell) was combined with PBMC diluted to 1×10⁸ cells/mL and incubated at 25° C. for 10 min. The cells were then diluted to 5×10⁷ cells/mL by combining them with an equal volume of 20 μg/mL common-capture RAM-ferrofluid (final [RAM-ferrofluid]=10 μg/mL), and the mixture was subjected to intermittent magnetic field gradients via cycles of exposure to a magnetic field gradient for 10 s with subsequent brief agitation for a total period of 10 min. The cells were then diluted to 3×10⁷ cells/mL and placed in a quadrupole magnetic separator for 15 min. At the end of the separation, the non-magnetic cell fraction was removed via Pasteur pipette aspiration, and fresh buffer (DPBS, 1% HSA) was added to the tube without resuspension of the magnetically collected cells and incubated with the magnetic cell fraction for 10 min. This process was repeated once more. After the final aspiration of the non-magnetic cell fraction, the sample was removed from the magnetic field gradient and the magnetic cell fraction was re-suspended in ImmunoCult XF T-cell expansion medium (STEMCELL Technologies) supplemented with 100 IU/mL IL-2 (Gibco) to a total concentration of 1×10⁶ cells/mL and incubated at 37° C. (5% CO₂). After an overnight incubation, 0.5 μg/mL mouse anti-human CD28 antibody of the IgG1 subclass (Mabtech) was added to the incubated cell fraction. The cells were periodically agitated and diluted to 1×10⁶ cells/mL with fresh expansion medium, and after 4 days in culture, the cells were analyzed for the presence of the CD25 marker via flow cytometry (FlowSight, Amnis). Upon analysis, 75% expressed the CD25 surface marker on Day 4, and cells experienced a 176-fold expansion by Day 15.

In a third method, 1 μg/mL anti-CD3 antibody of the IgG1 subclass (Ancell) was combined with PBMC diluted to 3×10⁷ cells/mL and incubated at 25° C. for 10 min. The cells were then diluted to 6×10⁵ cells/mL and centrifuged for 15 min at 300 rcf. The pellet was re-suspended at 1.4×10⁷ cells/mL and diluted to 7×10⁶ cells/mL by combining them with an equal volume of 20 μg/mL common-capture RAM-ferrofluid (final [RAM-ferrofluid]=10 μg/mL), and the mixture was subjected to intermittent magnetic field gradients via cycles of exposure to a magnetic field gradient for 10 s with subsequent brief agitation for a total period of 10 min. The cell mixture was then diluted to 3×10⁶ cells/mL and placed in a quadrupole magnetic separator for 15 min. At the end of the separation, the non-magnetic cell fraction was removed via Pasteur pipette aspiration, and fresh buffer (DPBS, 1% HSA) was added to the tube without resuspension of the magnetically collected cells and incubated with the magnetic cell fraction for 10 min. This process was repeated once more. After the final aspiration of the non-magnetic cell fraction, the sample was removed from the magnetic field gradient and the magnetic cell fraction was re-suspended in ImmunoCult XF T-cell expansion medium (STEMCELL Technologies) supplemented with 100 IU/mL IL-2 (Gibco) to a total concentration of 1×10⁶ cells/mL and incubated at 37° C. (5% CO₂). Following an overnight incubation, 0.5 μg/mL mouse anti-human CD28 antibody of the IgG1 subclass (Mabtech) was added to the incubated cell fraction. The cells were periodically agitated and diluted to 1×10⁶ cells/mL with fresh expansion medium, and after 4 days in culture, the cells were analyzed for the presence of the CD25 marker via flow cytometry (FlowSight, Amnis). Upon analysis, 55% expressed the CD25 surface marker on Day 4, and cells experienced a 104-fold expansion by Day 15.

Table 2 summarizes the results from the preceding three methods. It is apparent that the first modified labeling method, which includes simultaneous separation and activation, provided the best results in terms of activation and expansion. The other two methods were able to activate T cells and allowed for their expansion, but to a comparatively lesser extent. The significant increase in expansion seen with the first method was surprising and unexpected.

TABLE 2
Comparison of activation and expansion
using modified labeling methods.

| | % CD25+ | Expansion |
|---|---|---|
| Method Employed | on Day 4 | by Day 15 |
| Method 1 | 89% | 311-fold |
| Method 2 (no removal | 75% | 176-fold |
| of excess antibody) | | |
| Method 3 (excess | 55% | 104-fold |
| antibody removal) | | |

### EXAMPLE 2

### Effect of Nanoparticle to PBMC Ratio on Activation and Expansion

To test for the effect that the ratio of ferrofluid nanoparticles to PBMC has on T cell activation and expansion, cells were isolated and activated using the disclosed method, wherein the ratio of ferrofluid nanoparticles per cell was varied. PBMCs were isolated from human peripheral blood via the OptiPrep method (Axis Shield) by combining whole blood with 1.25 mL of OptiPrep per 10 mL of blood and centrifuging for 30 min at 1500 rcf (20° C.). The PBMC layer was removed and washed by centrifugation with Mg- and Ca-free DPBS (Sigma) containing 1% HSA to pellet the cells. The cell pellet was re-suspended and re-centrifuged at 300 rcf two more times to remove platelets.

6 μg/mL anti-CD3 antibody of the IgG1 subclass (Ancell) was combined with an equivalent volume of 40 μg/mL common-capture RAM-ferrofluid and vortexed. Equal volumes of PBMC at 1×10⁸ cells/mL, 8×10⁷ cells/mL, or 6×10⁷ cells/mL were added to three aliquots of the above mixture to yield final cell concentrations of 5×10⁷ cells/mL, 4×10⁷ cells/mL, or 3×10⁷ cells/mL, respectively (final [anti-CD3]=1.5 μg/mL; final [RAM-ferrofluid]=10 μg/mL). These concentrations correspond to ferrofluid nanoparticle per cell ratios of 60 nanoparticles/cell for the highest cell concentration, 75 nanoparticles/cell for the intermediate cell concentration, and 100 nanoparticles/cell for the lowest cell concentration. The mixtures were then subjected to intermittent magnetic field gradients via cycles of exposure to a magnetic field gradient for 30 s with subsequent brief agitation for a total period of 10 min. All samples were diluted (as necessary) to a cell concentration of 3×10⁷ cells/mL and the cell mixtures were placed in quadrupole magnetic separators for 15 min. At the end of the separation, the non-magnetic cell fractions were removed via Pasteur pipette aspiration, and fresh buffer (DPBS, 1% HSA) was added to the tubes without resuspension of the magnetically collected cells and incubated with the magnetic cell fractions for 10 min. This process was repeated once more. After the final aspiration of the non-magnetic cell fractions, the samples were removed from the magnetic field gradient and the magnetic cell fractions were re-suspended in ImmunoCult XF T-cell expansion medium (STEMCELL Technologies) supplemented with 100 IU/mL IL-2 (Gibco) to a total concentration of 1×10⁶ cells/mL and incubated at 37° C. (5% CO₂). The PBMC, the magnetic cell fractions, and the non-magnetic cell fractions were analyzed for the presence of the CD3 marker via flow cytometry (FlowSight, Amnis). After an overnight incubation, 0.5 μg/mL mouse anti-human CD28 antibody of the IgG1 subclass (Mabtech) was added to the incubated cell fractions. The cells were periodically agitated and diluted to 1×10⁶ cells/mL with fresh expansion medium, and after 4 days in culture, the cells were analyzed for the presence of the CD25 marker via flow cytometry. The results of this experiment are shown below in Table 3.

TABLE 3
Comparison of activation and expansion with
various nanoparticle to PBMC ratios.

| Nanoparticle: | % CD25+ | Expansion by |
|---|---|---|
| PBMC Ratio | on Day 3 | Day 15 |
| 60 | 60.5% | 100-fold |
| 75 | 56.5% | 87-fold |
| 100 | 49.8% | 51-fold |

### EXAMPLE 3

### Positive Selection of CD8+ T Cells with Simultaneous Activation and Subsequent Stimulation with Comparison to Dynabeads

To permit comparison between the embodiments described herein and Dynabeads Human T-Activator CD3/CD28, the latter of which requires purified T cells for optimal activation, cryopreserved CD8+ T cells were used for this experiment. To activate and stimulate the CD8+ T cells using Dynabeads, the manufacturer's recommended protocol was followed, with cells subsequently placed into ImmunoCult XF T-cell expansion medium (STEMCELL Technologies) supplemented with 100 IU/mL IL-2 (Gibco) to a total concentration of 1×10⁵ cells/mL and incubated at 37° C. (5% CO₂).

In contrast to the Dynabeads method, an experiment was performed as follows. 2.5 mL of 1 μg/mL monoclonal mouse-derived anti-human CD3 antibody of the IgG1 subclass (UCHT1 clone, Ancell) was combined with 2.5 mL of 8 μg/mL common-capture RAM-ferrofluid and briefly vortexed. The 5 mL mixture of antibody and ferrofluid was added to 5 mL of cells at 2×10⁷ cells/mL to yield a final cell concentration of 1×10⁷ cells/mL (final [anti-CD3]=0.25 μg/mL; final [RAM-ferrofluid]=2 μg/mL). These concentrations correspond to ferrofluid nanoparticle per cell ratios of 60 nanoparticles/cell. The mixture was then subjected to an intermittent magnetic field gradient via cycles of exposure to a magnetic field gradient for 30 s with subsequent brief agitation for a total period of 10 min. The mixture was placed in a quadrupole magnetic separator for 10 min. At the end of the separation, the non-magnetic cell fraction was removed, and fresh buffer (PBS, 1% HSA) was added to the tube with resuspension of the magnetically collected cells and incubated with the magnetic cell fraction for 10 min. This process was repeated twice more. After the final aspiration of the non-magnetic cell fraction, the sample was removed from the magnetic field gradient and the magnetic cell fraction (containing 99.7% of the CD8+ T cells) was re-suspended in ImmunoCult XF T-cell expansion medium (STEMCELL Technologies) supplemented with 100 IU/mL IL-2 (Gibco) to a total concentration of 1×10⁵ cells/mL and incubated at 37° C. (5% CO₂). After an overnight incubation, 0.5 μg/mL monoclonal mouse-derived anti-human CD28 antibody of the IgG1 subclass (3608-1-50, Mabtech) was added to the incubated cell fraction. The cells were periodically agitated and diluted to 1×10⁵ cells/mL with fresh expansion medium, and after 4 days in culture, the cells were analyzed for the presence of the CD25 marker via flow cytometry. Over a 10 day period, cells were counted to determine the average doubling time, and viability was assessed after 10 days. Although the Dynabead method and the method described in this example provided similar activation and expansion rates, it was found that the method described in this example provided cells that were more viable. For example, the viability of cells prepared according to the method described in this Example were 75% viable, whereas the viability of cells treated with Dynabeads was 62.5%. Accordingly, the presently described method provides for increased viability of cells, which could not have been predicted.

### EXAMPLE 4

### Comparison of Large-Scale and Small-Scale Positive Selection of T Cells from Leukapheresis Product with Simultaneous Activation and Subsequent Stimulation

Experiments were performed to demonstrate that positively selecting T cells with simultaneous activation and subsequent stimulation could be applied at large scale for a leukapheresis product. A leukapheresis product containing 2-2.5×10⁹ total nucleated cells was obtained from a commercial supplier (LE1003F, Stemexpress). For the small-scale experiment, the protocol from Example 3 was used, wherein antibody and common-capture ferrofluid were initially mixed, then combined with an equal volume of the leukapheresis product, and the intermittent magnetic field gradient and separation were carried out using a quadrupole magnetic separator. For the large-scale experiment, 50 mL of 1 μg/mL monoclonal mouse-derived anti-human CD3 antibody of the IgG1 subclass (UCHT1 clone, Ancell) was combined with 50 mL of 8 μg/mL common-capture RAM-ferrofluid and gently mixed briefly. The 100 mL mixture of antibody and ferrofluid was added to 100 mL of leukapheresis product contained within a blood bag at 2×10⁷ total nucleated cells/mL to yield a final cell concentration of 1×10⁷ total nucleated cells/mL (final [anti-CD3]=0.25 μg/mL; final [RAM-ferrofluid]=2 μg/mL). These concentrations correspond to ferrofluid nanoparticle per total nucleated cell ratios of 60 nanoparticles/total nucleated cell. The mixture was then subjected to an intermittent magnetic field gradient via cycles of exposure to a magnetic field gradient for 30 s by placing the bag onto a magnet array with subsequent brief agitation by inversion for a total period of 10 min. The mixture in the bag was placed onto a magnet array to separate for 10 min. At the end of the separation, the non-magnetic cell fraction was removed by pumping fresh buffer into the bag through an inlet, which forced the non-magnetic cell fraction to exit the bag through an outlet on the opposite side. While on the magnet array, the bag was then agitated to dislodge any non-magnetically labeled cells, after which fresh buffer was pumped into the bag to further wash the magnetically labeled cells. This process was repeated twice more. After the final removal of the non-magnetic cell fraction, the bag was removed from the magnetic field gradient and the magnetic cell fraction was re-suspended in ImmunoCult XF T-cell expansion medium (STEMCELL Technologies) supplemented with 100 IU/mL IL-2 (Gibco) to a total concentration of 5×10⁵ cells/mL and incubated at 37° C. (5% CO₂) in a shaker flask. Similarly, the magnetic cell fraction recovered from the small-scale experiment was re-suspended in ImmunoCult XF T-cell expansion medium (STEMCELL Technologies) supplemented with 100 IU/mL IL-2 (Gibco) to a total concentration of 5×10⁵ cells/mL and incubated at 37° C. (5% CO₂) in a shaker flask. After an overnight incubation, 0.5 μg/mL monoclonal mouse-derived anti-human CD28 antibody of the IgG1 subclass (3608-1-50, Mabtech) was added to the incubated cell fractions. After 4 days in culture, the cells were analyzed for the presence of the CD25 marker via flow cytometry, diluted to 1×10⁶ cells/mL with fresh expansion medium, and subsequently fed-batch cultured for another 13 days. Over the 17 day period, cells were counted to determine the expansion rate and viability was assessed. It was determined that the large-scale experiment gave nearly identical activation, expansion rate, and viability (80-90% at all time points) of expanded cells as the small-scale experiment.

### EXAMPLE 5

### Positive Selection of a T Cell Subset (i.e., CD4+ T Cells) from Leukapheresis Product with Simultaneous Activation and Subsequent Stimulation

As described herein, various embodiments allow for the simultaneous positive selection and activation of T cell subsets from a mixture of cells, with subsequent stimulation through the addition of one or more soluble co-stimulatory agents. The simultaneous separation and activation of CD4+ T cells is performed by first combining a biotinylated F(ab′)₂ fragment of mouse-derived anti-human CD4 of the IgG2a subclass (separation antibody) with a mouse-derived anti-human CD3 of the IgG1 subclass (activation antibody), wherein both antibodies are at a final concentration of 1 μg/mL. Next, a streptavidin-coated ferrofluid (average size of 130 nm) is combined with a poly(lactic-co-glycolic acid) nanoparticle (average size of 130 nm) coated with rat-derived anti-mouse IgG1, wherein both nanoparticles are at a final concentration of 8 μg/mL. The solution of two antibodies is then mixed with the solution of two nanoparticles at equal volume, and the resulting mixture is immediately combined with an equal volume of leukapheresis product at 2-2.5×10⁷ total nucleated cells/mL. The mixture is then subjected to intermittent magnetic field gradients via cycles of exposure to a magnetic field gradient for 30 s with subsequent brief agitation for a total period of 10 min. After the 10 min incubation period, a 15 min magnetic separation is performed to isolate the magnetically labeled CD4+ cells, where they are washed twice to remove non-magnetically labeled cells. The magnetically labeled cells are then recovered and re-suspended in ImmunoCult XF T-cell expansion medium (STEMCELL Technologies) supplemented with 100 IU/mL IL-2 (Gibco) to a total concentration of 1×10⁶ cells/mL and incubated at 37° C. (5% CO₂). Following an overnight incubation, 0.5 μg/mL mouse anti-human CD28 antibody of the IgG1 subclass (Mabtech) is added to the incubated cell fractions. The cells are periodically agitated and diluted to 1×10⁶ cells/mL with fresh expansion medium.

### EXAMPLE 6

### Negative Selection of T Cells from Leukapheresis Product with Simultaneous Activation and Subsequent Stimulation

As described herein, embodiments described herein provide for the simultaneous negative selection and activation of T cells from a mixture of cells, with subsequent stimulation through the addition of one or more soluble co-stimulatory agents. The simultaneous negative selection and activation of T cells is performed by first combining a cocktail of mouse-derived anti-human antibodies of the IgG1 subclass (anti-CD11b, anti-CD16, anti-CD19, anti-CD36, anti-CD41a, anti-CD56, and anti-CD235a; separation antibodies) with a biotinylated mouse-derived anti-human CD3 of the IgG2a subclass (activation antibody), wherein the activation antibody is at a final concentration of 1 μg/mL. Next, a ferrofluid (average size of 130 nm) coated with rat-derived anti-mouse IgG1 is combined with a streptavidin-coated poly(lactic-co-glycolic acid) nanoparticle (average size of 130 nm), wherein both nanoparticles are at a final concentration of 8 μg/mL. The solution of antibodies is then mixed with the solution of two nanoparticles at equal volume, and the resulting mixture is immediately combined with an equal volume of leukapheresis product at 2-2.5×10⁷ total nucleated cells/mL. The mixture is then subjected to intermittent magnetic field gradients via cycles of exposure to a magnetic field gradient for 30 s with subsequent brief agitation for a total period of 10 min. After the 10 min incubation period, a 15 min magnetic separation is performed to separate the magnetically labeled CD3− cells, and the non-magnetically labeled T cells are removed by aspiration; this can be repeated to ensure all magnetically labeled cells have been removed. The activated T cells are then recovered and re-suspended in ImmunoCult XF T-cell expansion medium (STEMCELL Technologies) supplemented with 100 IU/mL IL-2 (Gibco) to a total concentration of 1×10⁶ cells/mL and incubated at 37° C. (5% CO₂). Following an overnight incubation, 0.5 μg/mL mouse anti-human CD28 antibody of the IgG1 subclass (Mabtech) is added to the incubated cell fractions. The cells are periodically agitated and diluted to 1×10⁶ cells/mL with fresh expansion medium.

### EXAMPLE 7

### Negative Selection of a T Cell Subset (i.e., CD8+ T Cells) from Leukapheresis Product with Simultaneous Activation and Subsequent Stimulation

As described herein, embodiments described herein provide for the simultaneous negative selection and activation of CD8+ T cells from a mixture of cells, with subsequent stimulation through the addition of one or more soluble co-stimulatory agents. The simultaneous negative selection and activation of CD8+ T cells is performed by first combining a cocktail of mouse-derived anti-human antibodies of the IgG1 subclass (anti-CD4, anti-CD11b, anti-CD16, anti-CD19, anti-CD36, anti-CD41a, anti-CD56, anti-CD123, anti-CD235a, and anti-γδ TCR; separation antibodies) with a biotinylated mouse-derived anti-human CD3 of the IgG2a subclass (activation antibody), wherein the activation antibody is at a final concentration of 1 μg/mL. Next, a ferrofluid (average size of 130 nm) coated with rat-derived anti-mouse IgG1 is combined with a streptavidin-coated poly(lactic-co-glycolic acid) nanoparticle (average size of 130 nm), wherein both nanoparticles are at a final concentration of 8 μg/mL. The solution of antibodies is then mixed with the solution of two nanoparticles at equal volume, and the resulting mixture is immediately combined with an equal volume of leukapheresis product at 2-2.5×10⁷ total nucleated cells/mL. The mixture is then subjected to intermittent magnetic field gradients via cycles of exposure to a magnetic field gradient for 30 s with subsequent brief agitation for a total period of 10 min. After the 10 min incubation period, a 15 min magnetic separation is performed to separate the magnetically labeled CD8− cells, and the non-magnetically labeled CD8+ T cells are removed by aspiration; this can be repeated to ensure all magnetically labeled cells have been removed. The activated CD8+ T cells are then recovered and re-suspended in ImmunoCult XF T-cell expansion medium (STEMCELL Technologies) supplemented with 100 IU/mL IL-2 (Gibco) to a total concentration of 1×10⁶ cells/mL and incubated at 37° C. (5% CO₂). Following an overnight incubation, 0.5 μg/mL mouse anti-human CD28 antibody of the IgG1 subclass (Mabtech) is added to the incubated cell fractions. The cells are periodically agitated and diluted to 1×10⁶ cells/mL with fresh expansion medium.

The above specific descriptions are meant to exemplify and illustrate the embodiments and should not be seen as limiting the scope of the claims. Each and every referenced cited herein is incorporated by reference in its entirety and for its intended purpose.

1. A method of simultaneously separating and activating a population of T cells, or subsets thereof, the method comprising:
a) incubating a sample comprising a population of labeled magnetic particles, with at least one antibody that binds to a T-cell cell surface protein and activates the T cell, and a blood product;
b) applying a magnetic force to the sample;
c) separating the cells that are bound to the magnetic particles from the cells that are not bound to the magnetic particles,
wherein the labeled magnetic particles are labeled with a common-capture reagent.

2. The method of claim 1, further comprising culturing the cells that are bound to the magnetic particles in the presence of a soluble co-stimulatory agent.

3. The method of claim 2, wherein the co-stimulatory agent is anti-CD28, B7-1, B7-2, anti-CD2, LFA-3, or any combination thereof.

4. The method of claim 2, wherein the at least one soluble co-stimulatory agent is mouse-derived anti-human CD28 of the IgG1 subclass.

5. The method of claim 2, wherein the at least one soluble co-stimulatory agent is biotinylated.

6. The method of claim 5, wherein the at least one soluble co-stimulatory agent is biotinylated anti-human CD28, or fragments thereof.

7. The method of claim 2, wherein the co-stimulatory agent is not bound to a particle.

8. The method of claim 2, wherein the amount of the at least one soluble co-stimulatory agent can be independently varied with respect to the level of the at least one labeling antibody.

9. The method of claim 2, wherein the soluble co-stimulatory agent is a mixture of a mouse-derived anti-human CD28 of the IgG1 subclass and a mouse-derived anti-human CD2 of the IgG1 subclass.

10. The method of claim 2, wherein the soluble co-stimulatory agent is added at a single time point after the separating step.

11. The method of claim 2, wherein the one soluble co-stimulatory agent is added immediately after the separating step.

12. The method of claim 2, wherein the co-stimulatory agent is added about 1 minute to about 20 hours after the separating step.

13-15. (canceled)

16. The method of claim 1, wherein the labeled magnetic particles and the at least one antibody are mixed prior to being mixed with the blood product.

17. The method of claim 1, wherein the blood product is a whole peripheral blood product, a leukapheresis product, comprises mononuclear cells obtained from peripheral blood, comprises a population of enriched T cells at least one population of an enriched T cell subset, or is not a purified blood product.

18-27. (canceled)

28. The method of claim 1, wherein the at least one antibody binds to the magnetic particle or the at least one antibody binds to the common-capture reagent bound to the magnetic particle.

29. (canceled)

30. The method of claim 1, further comprising applying a magnetic force as an intermittent magnetic field gradient during the incubating step.

31. The method of claim 30, further comprising agitating the sample between the intermittent applications of the magnetic field.

32. The method of claim 1, further comprising applying a magnetic force as an intermittent magnetic field during the incubating step for about 10 seconds to about 30 seconds and optionally repeating the application of the magnetic force for a plurality of cycles.

33-39. (canceled)

40. A method of simultaneously separating and activating a population of T cells, or subsets thereof, the method comprising:
a) incubating a sample comprising:
a blood product;
a population of non-magnetic particles bound to at least one first antibody that binds to a T-cell surface protein and activates a T cell in the blood product;
a population of magnetic particles bound to at least one second antibody that binds to a cell surface protein of a cell in the blood product that is not a T cell, wherein the second antibody does not bind to the non-magnetic particles
b) applying a magnetic force to the sample;
c) separating the cells that are bound to the magnetic particles from the cells that are not bound to the magnetic particles; and
d) optionally culturing the cells that are not bound to the magnetic particles to expand the population of the cells.

41-58. (canceled)

59. A method of simultaneously separating and activating a sub-population of T cells, or subsets thereof, the method comprising:
a) incubating a sample comprising:
a blood product;
a population of magnetic particles bound to at least one first antibody that binds to a cell surface protein of a desired sub-population of cells in the blood product;
a population of non-magnetic particles bound to at least one second antibody that binds to and activates the desired sub-population of cells in the blood product, wherein the second antibody does not bind to the magnetic particles;
b) applying a magnetic force to the sample;
c) separating the cells that are bound to the magnetic particles from the cells that are not bound to the magnetic particles; and
d) optionally culturing the cells that are bound to the magnetic particles to expand the sub-population of the cells; or
the method comprises:
a) incubating a sample comprising:
a blood product;
a population of magnetic particles bound to at least one first antibody that binds to the cells not in a desired sub-population of cells in the blood product; and
a population of non-magnetic particles bound to at least one second antibody that binds to a cell surface protein of and activates the desired sub-populations of cells in the blood product, wherein the second antibody does not bind to the magnetic particles; and
b) applying a magnetic force to the sample;
c) separating the cells that are bound to the magnetic particles from the cells that are not bound to the magnetic particles; and
d) optionally culturing the cells that are not bound to the magnetic particles to expand the sub-population of the cells.

60-74. (canceled)
